(19) United States
(12) Patent Application Publication
(10) Pub. No.: US 2011/0282292 A1
(43) Pub. Date: Nov. 17, 2011

(54) PROTECTIVE MEDICAL DEVICE AND METHOD OF USE THEROF

(75) Inventors: Clayton R. Polowy, Scottsdale, AZ (US); Tearsin D. Shumway, Mesa, AZ (US); Darren Gray Jameson, Gilbert, AZ (US)

(73) Assignee: GRAY MEDICAL INNOVATIONS, LLC., Gilbert, AZ (US)

(21) Appl. No.: 12/780,729

(22) Filed: May 14, 2010

(51) Int. Cl. A61M 31/00

(52) U.S. Cl. 604/180; 604/288.01

(57) ABSTRACT

A guard for protecting an implantable port is provided. The guard includes a cover that is structured to protect a user's implantable port by substantially covering the port without the cover coming into contact with a user's skin above the port. The cover can be a rounded, triangular-shaped cover having peripheral edges that bow outwardly from the center to allow the cover to be placed over circular or triangular-shaped ports. The cover can also be releasably coupled to an adhesive base that is structured to adhere to the user's skin around the port, thus allowing the cover to remain in place on the user's skin. The guard may further include a housing to support the cover, the housing being coupled to the adhesive base prior to the adhesive base being adhered to the user's skin around the port. The cover may then be detachably connected to the housing.

### BACKGROUND OF THE INVENTION

1. Technical Field

This invention relates generally to medical devices, and in particular to a medical device for use with implantable ports.

2. State of the Art

An implantable port is often used to give chemotherapy treatment, intravenous antibiotics, and/or other medicines to both adults and children. The implantable port consists of a port cover (portal) and a thin, soft, plastic tube (catheter) connected thereto. The portal is surgically placed into the upper chest or arm of a patient and the free end of the catheter is usually inserted (tunneled) under the skin of the patient's chest with the tip of the catheter lying in a large vein (e.g., superior vena cava) just above the heart. This allows medicines, often chemotherapy treatments, to be given directly into the vein. The portal is typically a disc about 2.5-4 cm (1-1½ in) in diameter. Several identifying features of the implantable port include: a) the port cover; b) the palpation points (identifying features that help determine where percutaneous needle insertion should occur); c) the housing unit for the catheter; and d) the catheter itself.

Although the implanted port lies in the subcutaneous tissues of the patient just below the skin, the implanted port more often than not protrudes from the normal external plane of the patient's skin, the extent of which is dependant upon port placement by the medical professional, as well as the amount of subcutaneous fatty tissue, or lack thereof, present in the patient's tissues. Due to the aforementioned protrusions, and because a thin layer of skin lies superficial to the implanted port anterior to the patient's plane, the patient could be susceptible to several potential problems. For example, a potential problem to patients with the implantable port could be the use of an automobile's shoulder safety harness, or seat belt. The continual pressure and movement of the safety harness over the protruding port can be uncomfortable and could lead to chafing of the patient's skin over and around the port. To avoid this discomfort, a patient may unwisely choose to wear the seat belt incorrectly, by placing the shoulder harness behind his/her back, or sometimes not wear the seat belt at all, thus exposing himself/herself to additional potential dangers. Furthermore, abrasions in the skin above or around the implantable port could lead to the possibility of a port infection, which could, in some cases, lead to a blood infection that may require removal of the port. It is also important to note that most patients that receive implantable ports can be either immunocompromised or in danger of becoming immunocompromised.

In addition to the problems described above, normal everyday clothing can pose an additional problem to those persons that have an implantable port. For example, students or professionals that wear backpacks to and from school or work, respectively, persons who carry a purse by placing the shoulder strap on their shoulder, and persons that wear a bra, bathing suit, or other similar article of clothing can be vulnerable to the straps of these articles of apparel snagging, catching, or rubbing against the implantable port, which could lead to possible damage of the port, damage to tissue surrounding the port, and possible displacement and/or detachment of port-to-tissue connector sites. In addition, parents of a young child, or infant, who wish to hold the child close and rest the child's head against their chest, often need to be concerned about the placement of the child's head so that the child's head does not hit the implantable port and injure either the parent or child.

There is thus a need in the medical field to address the potential problems described above with respect to implantable ports.

### SUMMARY OF THE INVENTION

The present invention relates to medical devices, and more particularly to a medical device that provides protection from the damage caused by a patient's implantable port being rubbed or inadvertently bumped.

The present invention provides for a guard for an implantable port including a cover that is structured to be placed over a user's implantable port to substantially cover the protruding portion of the implantable port without the cover coming into contact with a user's skin above the implantable port. Such a cover provides protection for a user's implantable port against unwanted abrasion and contusion.

One aspect of the present invention may include the guard having an adhesive base with a hollow center portion therein. The cover may be releasably coupled to the adhesive base, and the adhesive base is structured so as to releasably adhere to the user around the implantable port, allowing the cover to substantially cover the implantable port without the cover contacting the user's skin above the implantable port.

Another aspect of the present invention may include the cover having a rounded shape with a peripheral edge defining the outer circumference of the rounded cover. Also, the cover may have an opening in the peripheral edge, the opening being placed over the catheter portion of the implantable port to prevent restriction of flow through the catheter. The cover may also have a lip extending at an angle from the peripheral edge, wherein the lip releasably couples to the user around the implantable port. The invention may further include a top surface of the rounded cover and a hole in the rounded cover, wherein the top surface is substantially flat.

Another aspect of the present invention may include the cover having a rounded triangular shape, the triangular shape having a peripheral edge that defines the outer circumference of the cover, the peripheral edge bowing outwardly from the center of the triangle to allow the cover to be placed over a circular or triangular implantable port.

Another aspect of the present invention may include the adhesive base being comprised of several layers, including a topside layer of woven fabric, an underside layer of woven fabric and a bottom layer of adhesive. A portion of the topside layer can be fixedly coupled to a portion of the underside layer so as to leave a remaining portion of both the topside layer and underside layer unconnected to each other. Such a structure allows the remaining portion of the topside layer to be placed on and couple to an upper side of the lip and the remaining portion of the underside layer to be placed under and couple to a bottom side of the lip to releasably couple the lip of the cover to the adhesive base. The adhesive layer may be placed on the bottom of the underside layer to releasably adhere the adhesive base to the user. Moreover, the adhesive layer may be, for example, a gel-based, liquid-based, epoxy, spray-on, tape, or strip adhesive.

Another aspect of the present invention may include the rounded cover having a top surface that is substantially flat. The top surface may have holes placed therein for aeration. Alternatively, the holes may be placed in the cover, and not in the top surface. Further in the alternative, the holes may be placed in both the top surface and not in the top surface. The present invention may also include the lip extending from the peripheral edge of the cover at an angle that is parallel with the top surface.

Another aspect of the present invention may include a cushioning layer fixedly coupled to the underside of the lip, wherein the cushioning layer is positioned between the lip and the remaining portion of the underside layer when the cover is coupled to the adhesive base.

Another aspect of the present invention may include a housing for holding the cover, the housing including a triangular-shaped frame defining a hollow center portion therein, an outer flange extending outwardly from the frame, an inner flange extending inwardly from the frame, a gap in the frame, and a plurality of projections extending inwardly from the frame and above the inner flange. The housing is structured to be placed around the implantable port and does not contact the skin over the implantable port. This allows the cover to protect the implantable port by being releasably coupled to the housing such that while the cover covers the implantable port, the cover does not contact the skin over the implantable port.

Another aspect of the present invention may include the cover being releasably coupled to the housing by placing the lip of the cover between the projections on the housing and the inner flange of the housing to secure the lip between the projections and the inner flange.

Another aspect of the present invention may include a w-shaped nose portion in a front end of the cover where the peripheral edge contacts the lip and a w-shaped receiving portion in the housing for receiving the w-shaped nose portion of the cover. Docking tabs may extend from the lip at a back end of the cover. Elastic members may be coupled to the frame between the projections and the inner flange with at least one elastic member being positioned on a front end of the frame under the w-shaped receiving portion to deform under the condition that the lip on the front end of the cover is pressed into the elastic member. Another elastic member may be positioned in the frame on a back end of the frame to deform under the condition that the docking tab on the back end of the cover is pressed into this elastic member.

Another aspect of the present invention may include the adhesive base being releasably coupled to the housing such that the hollow center portion of the housing overlaps the hollow center portion of the adhesive base with the adhesive base being structured to releasably adhere to the user around the implantable port such that implantable port is positioned within the housing. The remaining portion of the topside layer, as described above, is structured to be placed on and couple to the outer flange of the housing and the remaining portion of the underside layer, as described above, is structured to be placed under and couple to the housing to releasably couple the housing to the adhesive base. This allows the adhesive base to secure the housing, which in turn supports and secures the cover so that both the housing and cover protect the implantable port but do not contact the skin of the user over the implantable port.

Another aspect of the present invention may include another cushioning layer to also be fixedly coupled to the underside of the housing, wherein the cushioning layer is positioned between the housing and the remaining portion of the underside layer under the condition that the housing is coupled to the adhesive base.

Another aspect of the present invention may include a method of protecting an implantable port including placing a cover over the implantable port to protect the implantable port, and securing the cover over the implantable port.

Another aspect of the present invention may include placing a detachable housing around the implantable port, coupling the cover to the detachable housing, and adhering the detachable housing to the user's skin around the implantable port such that neither the detachable housing nor the cover contacts the user's skin above the implantable port.

Another aspect of the present invention may include coupling an adhesive base to a detachable housing, orienting the adhesive base and the detachable housing coupled thereto around the implantable port so that the adhesive base and the detachable housing do not contact the user's skin above the implantable port, coupling the cover to the detachable housing, and adhering the adhesive base to the user's skin around the implantable port such that none of the adhesive base, the detachable housing, or the cover contacts the user's skin above the implantable port.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a top view of a port guard in accordance with an embodiment of the present invention.

FIG. 2 is a cross-sectional view of the port guard taken at the line 2-2 in FIG. 1 in accordance with the present invention.

FIG. 3 is a top view of an adhesive base in accordance with the present invention.

FIG. 4 is a cross-sectional view of the adhesive base taken at line 4-4 in FIG. 3 in accordance with the present invention.

FIG. 5 is a top view of the port guard and adhesive base in accordance with the present invention.

FIG. 6 is a cross-sectional view of the port guard and adhesive base taken at line 6-6 in FIG. 5 in accordance with the present invention.

FIG. 7 is a top view of the port guard in accordance with an embodiment of the present invention.

FIG. 8 is a perspective view of the port guard in accordance with an embodiment of the present invention.

FIG. 9 is a top view of a frame in accordance with an embodiment of the present invention.

FIG. 10 is a cross-sectional view of the frame taken at line 10-10 in FIG. 9 in accordance with an embodiment of the present invention.

FIG. 11 is a cross-sectional view of the frame taken at line 11-11 in FIG. 9 in accordance with an embodiment of the present invention.

FIG. 12 is a cross-sectional view of the frame taken at line 12-12 in FIG. 9 in accordance with an embodiment of the present invention.

FIG. 13 is a top view of the port guard, frame, and adhesive base in accordance with an embodiment of the present invention.

FIG. 14 is a cross-sectional view of the port guard, frame, and adhesive base taken at line 14-14 in FIG. 13 in accordance with an embodiment of the present invention.

### DETAILED DESCRIPTION OF EMBODIMENTS OF THE INVENTION

As discussed above, embodiments of the present invention relate to a medical device for use in conjunction with an implantable port.

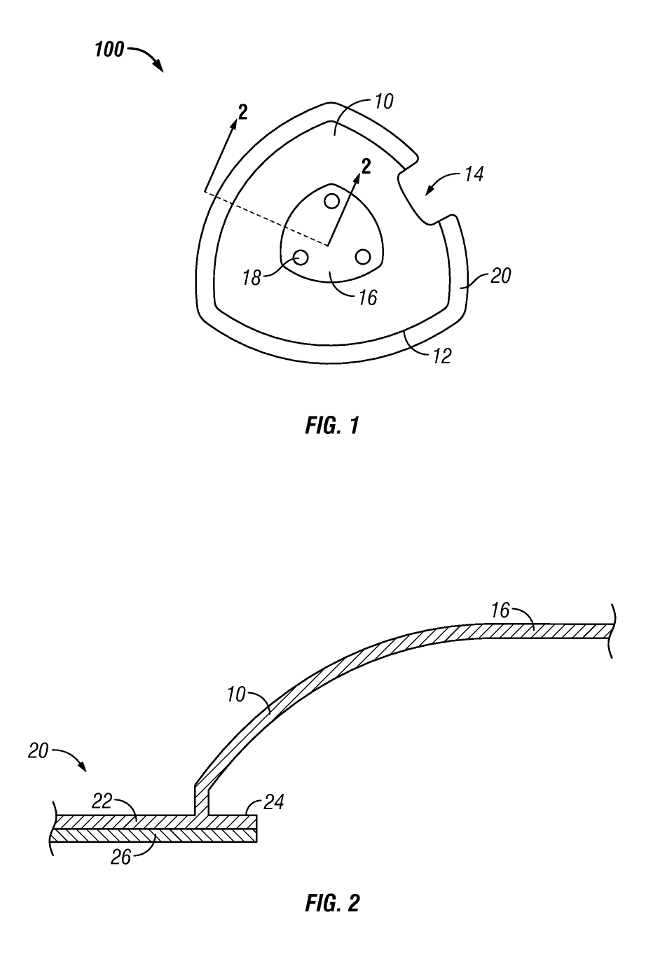

As shown in FIG. 1, the guard 100 includes a cover portion 10, a peripheral edge 12 defining the base of the cover portion 10, and a lip 20 extending at an angle from the peripheral edge 12. The cover portion 10 is structured to generally have a convex, hollow shape to thus allow a wearer, or user, of an implantable port to place the hollowed-out portion of the convex shape of the guard 100 over the port to protect the port without the cover portion 10 of the guard 100 contacting the wearer's skin on top of the port. In certain embodiments, the hollow shape of the cover portion 10 may have more of a domed, triangular shape whose peripheral edge 12 bows outward from the center of the triangular shape, which allows the guard 100 to be placed over implantable ports of either circular or triangular-shaped varieties without the guard 100 contacting the user's skin above the port.

The peripheral edge 12 of the cover portion 10 defines the base of the cover portion 10, or, in other words, the outer circumference of the cover portion 10. The peripheral edge 12 runs along the entire outer circumference of the cover portion 10, except for an opening 14 in a side of the cover portion 10. The opening 14 in the cover portion 10, and thus the peripheral edge 12, permits the guard 100 to be placed over the implantable port with the opening 14 above the port-to-catheter connection so as to not constrict, or occlude, the flow through the catheter. For example, by placing the opening 14 above the port-to-catheter connection, the catheter does not receive any stress or pressure from the guard 100 itself being placed on the user's skin around the port and the catheter will be protected from sudden increases in force or pressure resulting from the guard 100 being bumped or otherwise jogged while the guard 100 is on the user's skin. Consequently, the opening 14 allows the guard 100 to protect the implantable port and the user's skin above the implantable port, while at the same time preventing the catheter from being occluded.

The lip 20 of the guard 100 extends at an angle from the peripheral edge 12 of the cover portion 10. In certain embodiments, the lip 20 extends at an angle that allows the entire underside portion of the lip 20 to contact the user's skin under the condition that the guard 100 is placed directly on the user's skin. The lip 20 is relatively uniform in width and runs along the entire peripheral edge 12. Accordingly, the lip 20 does not extend or run into the opening 14.

The cover portion 10 may also include a top portion 16 that is substantially flat. The edges of the top portion 16, where the cover portion 10 meets the top portion 16, are generally rounded, or soft, as opposed to being harsh edges. This allows materials or articles that brush up against, or collide with, the guard 100 to slide and slip across the cover portion 10 and the top portion 16 without snagging or catching thereon. The top portion 16 may also have holes 18 therein. The holes 18 allow the guard 100 to “breathe,” in that the holes 18 serve to aerate the guard 100 so that moisture and wetness do not remain within the guard 100 under the condition that the guard 100 is adhered to the user's skin. In other embodiments, the holes 18 may be placed in the cover portion 10 only, or in a combination of the cover portion 10 and the top portion 16. The holes 18 are generally circular in shape for ease of manufacturing, but the holes 18 may take any shape or form that still allows the guard 100 to aerate when placed against the user's skin. For example, the holes 18 may be shaped as squares, triangles, lines, or other geometric shapes. Further, the holes 18 may be made into, for example, the initials of the user, the shape of the user's favorite animal, or another shape preferred by the user.

As shown in FIG. 2, a portion of the lip 20 that extends outwardly from the cover portion 10 is an outer lip portion 22. The lip 20 also has an inner lip portion 24 that extends inwardly from the cover portion 10. The inner lip portion 24 is smaller in width than the width of the outer lip portion 22. Underneath the lip 20, a cushioning layer 26 may be fixedly coupled to the lip 20. Specifically, the cushioning layer 26 is positioned below the entire lip 20, including each of the inner lip portion 24 and the outer lip portion 22. The cushioning layer 26 is made of any medically-approved material that is latex-free and serves to provide a degree of compliancy, or give, when pressure is applied to the cover portion 10. When added pressure is applied to the cover portion 10 by the guard 100 being bumped, that pressure is transferred through the rigid structure of the cover portion 10 into the lip 20 and against the user's skin. Thus, the cushioning material 26 is structured to deform under the lip 20 in response to any added pressure or force being applied to the cover portion 10, such that the pressure or force felt by the user against the user's skin is reduced, if not eliminated. The degree of give in the cushioning material 26 also protects the guard 100 from excessive damage, because as the guard 100 receives a strike or hit, the give in the cushioning material 26 absorbs the blow and allows the guard 100 to withdraw from or redirect the effects of the blow.

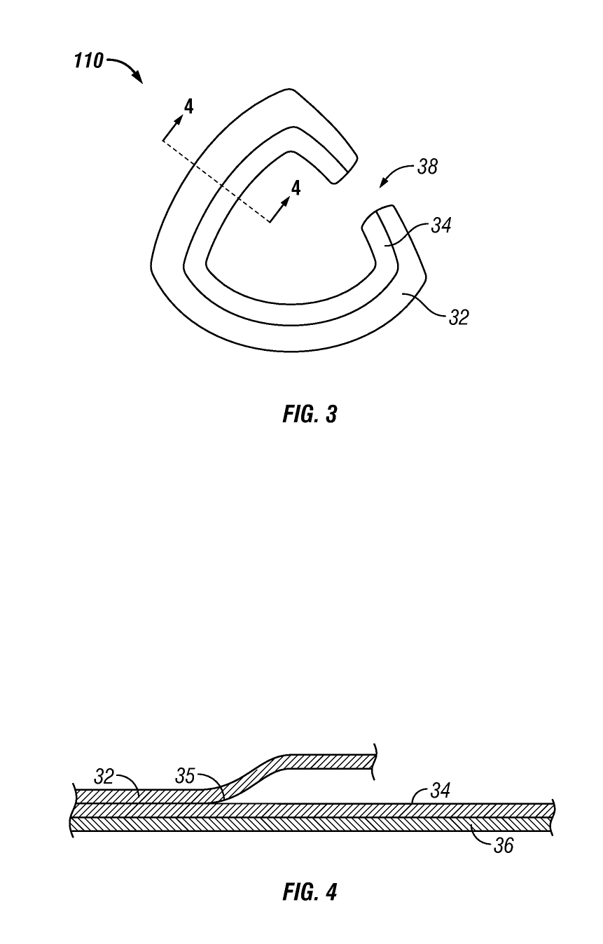

As shown in FIG. 3, an adhesive base 110 may be used to adhere the guard 100 to the user. To do so, the guard 100 is first coupled to the adhesive base 110, which is then adhered to the skin of the user around the implantable port. To facilitate the coupling of the adhesive base 110 to the guard 100, the adhesive base 110 is structured in the same shape as the peripheral edge 12 and the lip 20 of the guard 100. This is because the peripheral edge 12 and the lip 20, extending from the peripheral edge 12, are the portions of the guard 100 that will come into contact with the adhesive base 110. Moreover, because the guard 100 has a hollow, or rounded, center portion, defined by the convex shape of the cover portion 10 that is placed over the implantable port, the adhesive base 110 must include an open center portion therein to fit around the implantable port. The open center portion of the adhesive base 110 is defined by the structure of a bottom layer 34 of the adhesive base 110.

The bottom layer 34 is generally a thin strip of material that, as mentioned above, mimics the shape of the peripheral edge 12. The width of the bottom layer 34 is larger than the width of the lip 20 of the guard 100. Once the material of the bottom layer 34 is positioned, or formed, to mimic the shape of the peripheral edge 12, an open space between respective sides of the bottom layer 34 is created, the open space being the open center portion of the adhesive base 110, mentioned above. The implantable port fits therein. In other words, with an open center portion in the adhesive base 110, the adhesive base 110 can be placed against the user's skin and around the implantable port without contacting the implantable port, or the skin of the user above the implantable port, because the implantable port fits within the open center portion of the adhesive base 110.

The adhesive base 110 includes a top layer 32 in addition to the bottom layer 34. Each of the top layer 32 and bottom layer 34 may be made of woven fabric material, fibers embedded into cloth, or any other material that is flexible, yet durable. The top layer 32 is positioned above and overlays the bottom layer 34. The width of the top layer 32 is less than the width of the bottom layer 34. The outer edges of each of the top layer 32 and bottom layer 34 are aligned. Thus, the inner edges of each of the top layer 32 and bottom layer 34 are not aligned. As shown in FIG. 4, a portion of the top layer 32 from the outer edge of the top layer 32 to a point 35 inward from the outer edge is fixedly coupled to a portion of the bottom layer 34 from the outer edge of the bottom layer 34 to the inward point 35. From the point 35 to the inner edges of each of the top layer 32 and the bottom layer 34, the top layer 32 and the bottom layer 34 are not coupled together.

As shown in FIG. 3, the adhesive base 110 also includes a gap 38, in a side portion thereof. In certain embodiments where the shape of the adhesive base 110 is a triangle, the gap 38 is positioned in one of the three sides of the triangular shape. The width of the gap 38 is similar in size to the width of the opening 14 in the guard 110 and serves a similar function. Specifically, the gap 38 permits the adhesive base 110 to be placed over the implantable port with the gap 38 over the port-to-catheter connection so as to not constrict, or occlude, the flow through the catheter.

The width of the top layer 32 can be uniform on all sides of the adhesive base 110, and the width of the bottom layer 34 can also substantially uniform on all sides. However, in embodiments where the adhesive base 110 is triangular in shape, the widths of each of the top layer 32 and bottom layer 34 on a third side of the triangle shape can be smaller than the respective widths of the top layer 32 and bottom layer 34 on the other two remaining sides. For example, in certain embodiments, the width of the bottom layer 34 may be approximately 14 mm on the third side of the triangle shape and 17 mm on the two remaining sides. Moreover, in such a configuration, the gap 38 is positioned on the third side having the smaller widths because it provides at least one advantage to the user. Specifically, the port-to-catheter connection and catheter are generally placed under a user's skin between the port and the user's neck and they point to the user's neck. Also, when a user wears clothing the port may sometimes protrude, or nearly protrude, from underneath the collars of the user's clothing. Protrusion of the port from underneath the user's clothing can be embarrassing to the user. Thus, to avoid having the adhesive base 110 protrude out from under a user's clothing, the adhesive base 110 is structured to have the gap 38 positioned in the third side of the adhesive base 110 having the top layer 32 and the bottom layer 34 with the smallest widths. This is to allow not only the gap 38 to be placed over the port-to-catheter connection and catheter (that lies closest to the neck, as described above), but to place the side of the adhesive base 110 with the shortest width nearest the user's neck to prevent the adhesive base 110 from peaking out from under the user's clothes.

As also shown in FIG. 3, an adhesive layer 36 may be placed underneath the bottom layer 34. Accordingly, the adhesive layer 36 may be formed or manufactured in the same shape and configuration as the shape of the bottom layer 34. The adhesive layer 36 serves to adhere the adhesive base 110 to the user's skin around the implantable port. The adhesive layer 36 may be, for example, a gel-based, liquid-based, epoxy, spray-on, tape, or strip adhesive. A gel-based adhesive layer may be useful in allowing the user to apply, remove, and reapply the adhesive layer 36 as often as necessary to remove or attach the adhesive base 110 to his/her skin. The adhesive strength of the adhesive layer 36 must be strong enough to prevent the guard 100 from being dislodged from the user's skin from, for example, bumps to, hits on, and abrasions of, the guard 100. Yet, the adhesive strength must not be so strong as to prevent the user from removing the adhesive base 110, with the guard 100 coupled thereto, on his/her own.

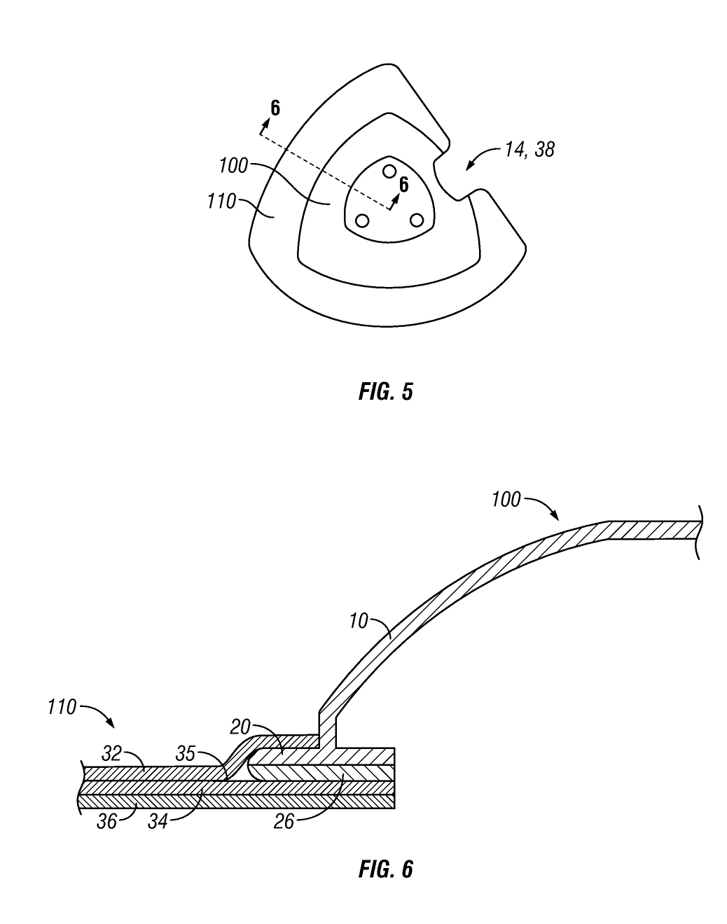

As shown in FIGS. 5 and 6, and as mentioned above, the guard 100 can be releasably coupled to the adhesive base 110. The adhesive base 110 may be adhered to the user's skin around the implantable port. As shown in FIG. 5, the guard 100 is placed in the center of the adhesive base 110. The opening 14 in the guard 100 and the gap 38 in the adhesive base 110 align, and the top layer 32 of the adhesive base 110 overlaps the lip 20 of the guard 100.

As shown in FIG. 6, the top layer 32 of the adhesive base 110 is structured to overlay and couple to the outer lip portion 22 of the lip 20. The bottom layer 34 of the adhesive base 110 is structured to underlay and couple to the underside of the lip 20, or, if the cushioning layer 26 is coupled to the underside of the lip 20, the bottom layer 34 of the adhesive base 110 is structured to underlay and couple to the cushioning layer 26. The guard 100 and the adhesive base 110 can be coupled together by, for example, stitching, glue, adhesives, hook-and-loop fasteners, staples, two-sided tape, or the like. By placing the lip 20 of the guard between the layers 32 and 34 of the adhesive base 110, as described above and shown in the figures, the guard 100 is securely coupled to and within the adhesive base 110. Also, the cushioning layer 26, the bottom layer 34, and the adhesive layer 36 are each structured to extend to at least the inner edge of the inner lip portion 24 of the lip 20. Such a structure prevents the lip 20 from gouging or otherwise contacting the user's skin.

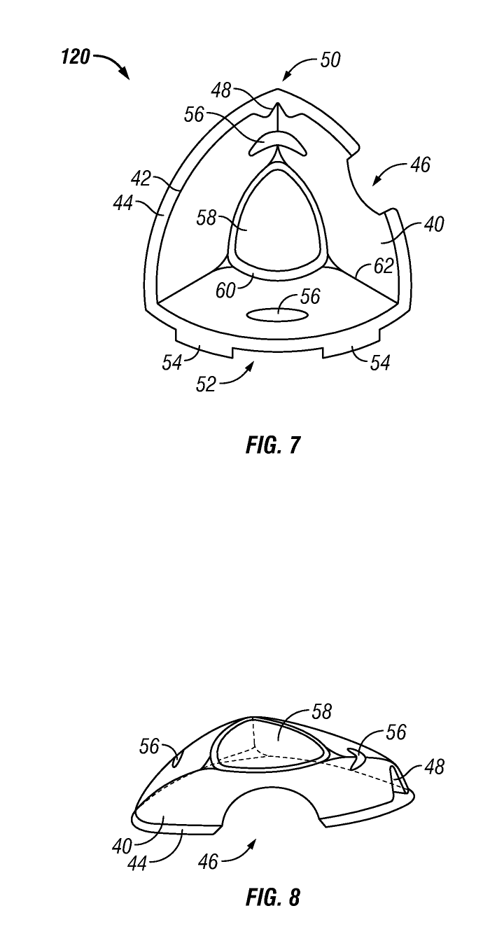

As shown in FIG. 7, an additional embodiment of the present invention includes a port guard 120 having a cover portion 40, a peripheral edge 42, a lip 44, and a top portion 58. The cover portion 40, the peripheral edge 42, the lip 44, and the top portion 58 are structured similarly to the cover portion 10, the peripheral edge 12, the lip 20, and the top portion 16 of the guard 100, respectively. Thus, a detailed explanation of the structure of the cover portion 40, the peripheral edge 42, the lip 44, and the top portion 58 is not provided. However, any differences between these features will be explained in greater detail below.

In certain embodiments where the port guard 120 is shaped in a rounded triangle, the top portion 58 further includes a rounded edge 60 where the top portion 58 meets the cover portion 40. The rounded edge 60 allows any material that brushes up against or passes over the port guard 120 to do so without snagging on the rounded edge 60. Moreover, the top portion 58 is shaped similarly to the triangular shape of the port guard 120. Also, the cover portion 40 includes ridges 62 running from the vertices of the triangular-shaped port guard 120 to the vertices of the triangular-shaped top portion 58. The ridges 62 effectively divide the cover portion 40 into three distinct sides. As shown if FIG. 8, the ridges 62 and the sides of the cover portion 40 are rounded for the same reasons that edge 60 is rounded—any material that brushes up against or passes over the sides of the cover portion 40 and the ridges 62 do so without snagging on either the sides of the cover portion 40 or the ridges 62.

Referring again to FIG. 7, the port guard 120 also includes an opening 46 in one of the sides of the cover portion 40 and thus the lip 44. The opening 46 is structured to be placed over the port-to-catheter connection and/or the catheter itself that lie underneath the user's skin. The opening 46 therefore allows the port guard 120 to be placed against the user's skin around the implantable port to cover and protect the implantable port and yet keep the port guard 120 from blocking, crimping, or otherwise applying pressure to the port-to-catheter connection or the catheter that might damage the catheter, impede the flow through the catheter, or irritate the skin over the port-to-catheter connection or catheter. As shown in FIG. 8, the opening 46 is generally semi-circular, or arc-like, but may be of any shape and size suitable to prevent the port guard 120 from unnecessarily contacting the port-to-catheter connection or the catheter, as described above.

As also shown in FIG. 7, the port guard 120 includes a front end 50 and a back end 52. The front end 50 of the port guard 120 includes a w-shaped, or double-grooved, nose portion 48. The w-shaped nose portion 48 is structured to be part of both the peripheral edge 42 and the ridge 62 in the front end 50 and sits just above the lip 44. The w-shaped nose portion 48 is configured to engage the w-shaped receiving portion 78, to be described in greater detail below.

The port guard 120 further includes a docking tab 54. The docking tab 54 is structured to protrude at an angle from the lip 44 at the back end 52 of the port guard 120. The docking tab 54 allows the back end 52 of the port guard 120 to releasably engage and couple to a housing 130, to be described in greater detail below. Alternatively, docking tab 54 may comprise a plurality of docking tabs 54 that protrude, or extend, at an angle from the lip 44 at the back end 52. In certain embodiments, docking tab 54, or docking tabs 54, protrudes from the lip 44 at the same angle that the lip 44 protrudes, or extends, from the peripheral edge 42. Docking tab 54 may be positioned on the lip 44 near the center of the port guard 120 at the back end 52, whereas the plurality of docking tabs 54 may be positioned along the lip 44 at predetermined intervals at the back end 52.

The port guard 120 further includes holes 56 that allow the port guard 120 to “breathe,” similar to holes 18 described above. However, unlike the holes 18, the holes 56 serve a dual purpose. Not only do the holes 56 aerate the port guard 120 so that moisture and wetness do not remain within the port guard 120 under the condition that the port guard 120 is adhered to the user's skin, but the holes 56 allow a user to place his/her fingers into the holes 56 to grip the port guard 120 and pinch, or otherwise deform the port guard 120 to remove the port guard 120 from the housing 130. Thus, the holes are shaped to receive the tips of the user's fingers and provide grip to the user's fingers for removing the port guard 120 from the housing 130, to be described below. The holes 56 can be placed in the cover portion 40 on each of the front end 50 and back end 52 of the port guard 120, the hole 56 in the front end 50 is generally located above the w-shaped nose portion 48 and the hole 56 in the back end is generally centered on the side of the cover portion 40 at the back end 52.

As shown in FIG. 8, the port guard 120 has a thin profile when viewed from the side. In certain embodiments of the port guard 120, the rounded cover portion 40 has a height measuring approximately 6-7 mm from a center point under the top portion 58 on a horizontal plane created by the base of the point of the port guard 120 to the top of the top portion 58. Also, the triangular-shaped port guard 120 may measure 4 cm×4 cm×4 cm from rounded vertex to rounded vertex at the base of the cover portion 40. In other words, the length of a side of the triangular-shaped port guard 120 measures approximately 4 cm from one vertex to another vertex at the base of the cover portion 40 where the cover portion 40 meets the peripheral edge 42. In addition, the triangular-shaped top portion 58 may measure 2 cm×2 cm×2 cm from rounded vertex to rounded vertex. In other words, the length of a side of the triangular-shaped top portion 58 measures approximately 2 cm from one vertex to another vertex at the rounded edge 60 where the cover portion 40 meets the top portion 58.

The port guard's 120 thin profile allows materials and fabrics that come into contact with the port guard 120 to slide and slip over the port guard 120 with little or no resistance from the port guard 120 and with little or no impact on the implantable port. As fabrics and materials more easily slide over the port guard 120, instead of catching or snagging on the port guard 120, less force is generated by the materials on the port guard 120 and thus less impact is transferred through the port guard 120 to the user's skin to which the port guard 120 is coupled. All the while, the implantable port is protected from direct contact by the materials. Such a configuration therefore not only protects the implantable port and the skin surrounding the implantable port from contusion and abrasion, but also prevents the port guard 120 from being unnecessarily tugged or pulled by the user's everyday clothing. This provides a convenience and security to the user by allowing the user to return to a normal, active lifestyle without worrying about the user's own clothing damaging the implantable port or the surrounding skin.

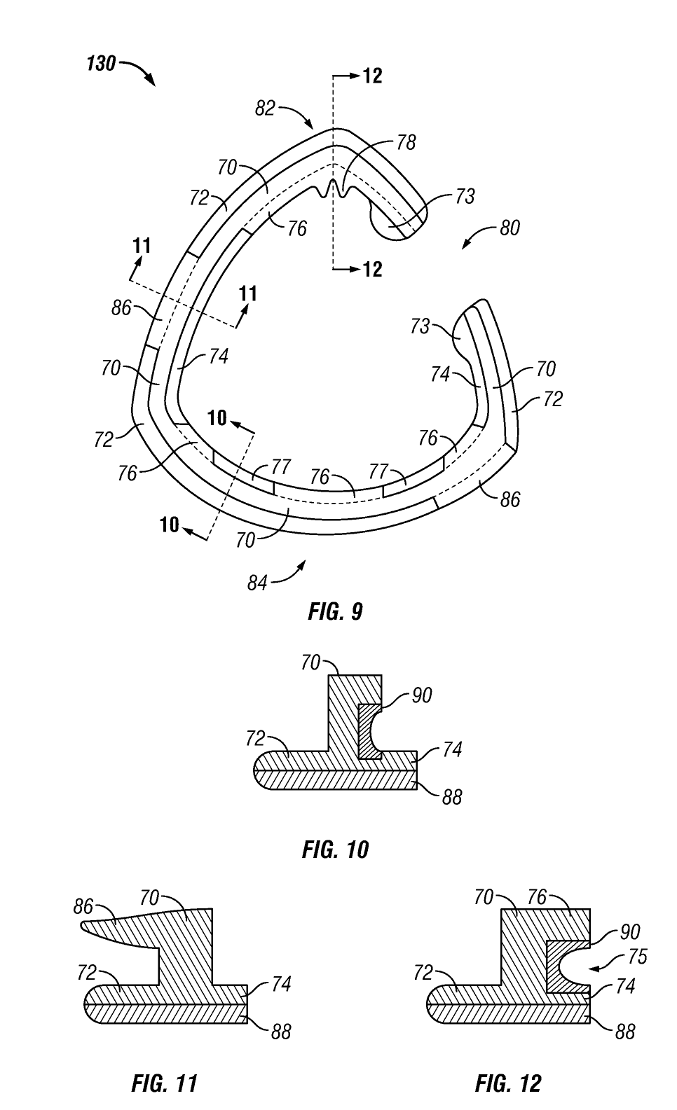

As shown in FIG. 9, the housing 130, mentioned above, is structured to be placed on the user's skin around the user's implantable port. The housing 130 includes a frame 70, an outer lip 72, and an inner lip 74. The outer lip 72 and the inner lip 74 extend outwardly and inwardly, respectively, from the sides of the frame 70 at the base of the frame 70. Also, the frame 70 is usually structured in the same shape as the peripheral edge 42 of the cover portion 40 of the port guard 120 so that the frame 70 may receive the peripheral edge 42 and lip 44 of the port guard 120, such that the port guard 120 can be placed within the housing 130. The frame 70 thus defines a hollow center portion between respective sides of the frame 70. The implantable port is placed within the hollow center portion when the frame 70 is placed against the user's skin.

The frame 70 further includes protrusions 76 that extend inwardly from the sides of the frame 70 at the top of the frame 70 and extend substantially parallel with the inner lip 74 to create a groove 75 therebetween. The protrusions 76 are generally located on a front end 82 and a back end 84 of the housing 130. The front end 82 corresponds to the front end 50 of the port guard 120, and the back end 84 corresponds to the back end 52 of the port guard 120. When the port guard 120 is releasably coupled to the housing 130, the lip 44 of the port guard 120 fits into the groove 75 by resting on top of the inner lip 74 and under the protrusions 76.

The housing 130 further includes the w-shaped receiving portion 78, mentioned above. The w-shaped receiving portion 78 is located on one of the frame 70 or the protrusions 76 in the front end 82. The w-shaped, or double-grooved, receiving portion 78 is positioned on the housing 130 to receive the w-shaped nose portion 48 on the port guard 120. Specifically, under the condition that the port guard is releasably coupled to the housing 130, the w-shaped nose portion 48 fits into, or docks with, the w-shaped receiving portion 78 in the housing 130. Such a configuration prevents the port guard 120 from moving or shifting within the housing 130 once the w-shaped portions 48 and 78 are docked together.

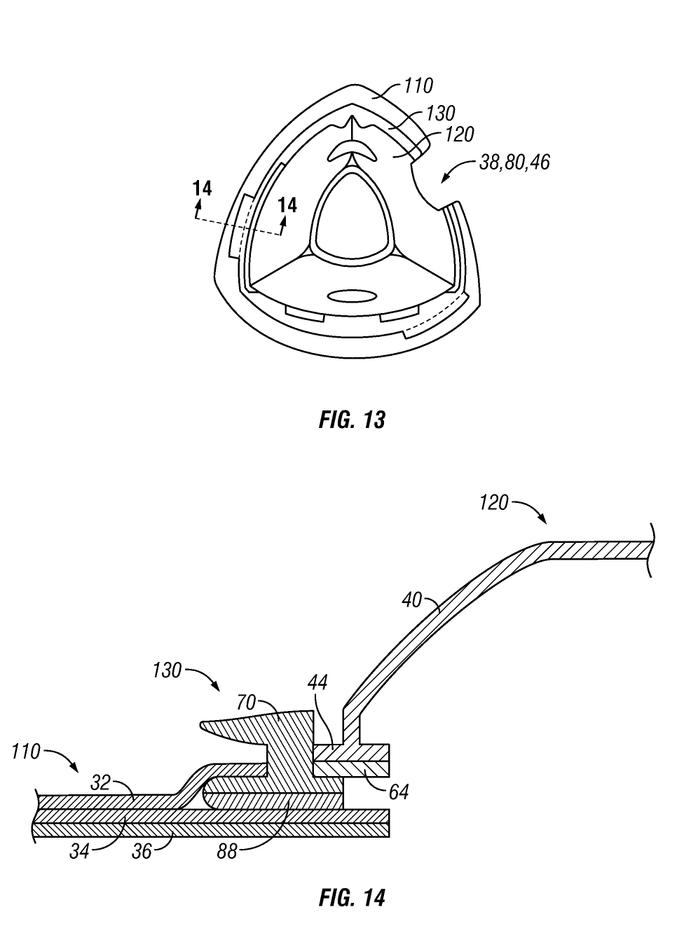

The housing 130 also includes a channel 80 that is positioned in one of the sides of the housing 130. The channel 80 runs through the entire frame 70, such that the frame 70 is open on one side and is not an enclosed triangle, as shown in FIG. 9. The channel 80 serves a similar purpose as the gap 38 in the adhesive base 110 and the openings 38 and 46 in the guard 110 and the port guard 120, respectively. The channel 80 allows the housing 130 to be positioned on the user's skin around the implantable port without interfering with the port-to-catheter connection or catheter under the user's skin. The channel 80 is thus positioned in the frame 70 such that under the condition the port guard 120 is coupled to the housing 130, the opening 46 in the port guard 120 overlaps the channel 80 in the housing 130, as shown in FIG. 13.

The inner lip 74 of the housing 130 also includes bulging portions 73 of the inner lip 74 where the inner lip 74 meets the channel 80. In other words, to provide a larger lip on which the port guard 120 may rest when coupled to the housing 130, the inner lip 74 is wider at the bulging portions 73 located where the inner lip 74 meets the channel 80, as shown in FIG. 9. The housing 130 also includes tabs 86 for assisting the user in securing the housing 130 while the user removes the port guard 120 from the housing 130. The tabs 86 extend outwardly from the frame 70 at the top of the side of the frame 70. The tabs 86 extend from the frame 70 equidistant to the extension of the outer lip 72, and the tabs 86 are not positioned on the side of the frame 70 that includes the channel 80. Indeed, one of the tabs 86 may be positioned in the center of the side of the frame 70 that does not include the channel 80 and does not form part of the back end 84 of the housing 130. Another of the tabs 86 may be positioned on the side of the frame 70 near the vertex of the frame 70 formed by the side of the frame containing the channel 80 and the side of the frame that forms part of the back end 84 of the housing 130. Such placement of the tabs 86 facilitates the removal of the port guard 120 from the housing 130.

The housing 130 further includes notches 77 in one of the frame 70 or the protrusions 76 that are wide enough to allow the docking tabs 54 of the port guard 120 to be placed therein. Under the condition that the port guard 120 is placed within the housing 130, the docking tabs 54 fit within the notches 77 and allow the docking tabs 54 to contact an elastic member 90 placed on the frame 70, as shown in FIG. 10. The elastic member 90 may also be located within the groove 75 under the w-shaped receiving portion 78, as shown in FIG. 12.

As shown in FIG. 10, which is a cross-section of the housing 130 at the line 10-10 in FIG. 9, the elastic member 90 is embedded in the inner side of frame 70 and a portion of the inner lip 74. The elastic member 90 may be formed in any thickness, size, and shape that allows the docking tab 54 to be placed thereagainst and deform to secure the docking tab 54 in place. The elastic member 90 may be made of non-latex rubber, or any other material that deforms under pressure, but is resilient enough to resist the deformation.

As shown in FIG. 12, which is a cross-section of the housing 130 at the line 12-12 in FIG. 9, the elastic member 90 is embedded under the w-shaped receiving portion 78 in the groove 75 created by the inner lip 74 and the protrusion 76. The elastic member 90 under the w-shaped receiving portion 78 may have a semi-circle shape to receive the lip 44 at the vertex on the front end 50 of the triangular-shaped port guard 120.

As shown in FIG. 11, which is a cross-section of the housing 130 at the line 11-11 in FIG. 9, the tab 86 is contoured to better facilitate the grip of the user's finger against the tab 86 when the user is removing the port guard 120 from the housing 130. Also, as shown in FIGS. 10-12, a cushion layer 88 may be placed on the underside of the frame 70, the inner lip 74, and the outer lip 72 of the housing 130. The cushion layer 88 may be adhered to the user's skin to couple the housing 130 to the user. The cushion layer 88 provides a level of comfort and cushioning to the user by preventing the rigid housing 130 from coming into contact with the user when the housing 130 is bumped or jarred.

As shown in FIGS. 13 and 14, the adhesive base 110, the port guard 120, and the housing 130 may be releasably coupled together. The adhesive base 110 can be releasably coupled to the housing 130, and the housing 130 may be releasably coupled to the port guard 120. Generally, the port guard 120 is not coupled to the adhesive base 110, but can be if needed, similar to the adhesive base 110 coupling to the guard 100, as described above. The adhesive base 110 is typically coupled to the housing 130 and the adhesive base 110 is adhered to the user's skin around the implantable port, such that the adhesive base 110 and the housing 130 surround the implantable port. Thereafter, the port guard 120 may be releasably coupled to the housing 130 to cover and protect the implantable port. Likewise, the port guard 120 may be decoupled from the housing 130 when desired. When the adhesive base 110, the port guard 120, and the housing 130 are coupled together the gap 38 in the adhesive base 110, the channel 80 in the housing 130, and the opening 46 in the port guard 120 overlap one another, as shown in FIG. 13.

As shown in FIG. 14, which is a cross-section of the combined adhesive base 110, the port guard 120, and the housing 130 taken along line 14-14 in FIG. 13, the adhesive base 110 can be coupled to the housing 130 by the top layer 32 of the adhesive base 110 being overlaid and coupled to the outer lip 72 of the housing 130. The bottom layer 34 of the adhesive base 110 is structured to underlie and couple to the underside of the outer lip 72, the frame 70 and the inner lip 74. Alternatively, if the cushion layer 88 is coupled to the underside of the outer lip 72, the frame 70 and the inner lip 74, the bottom layer 34 of the adhesive base 110 is structured to underlie and couple to the cushion layer 88. By placing the outer lip 72 of the housing 130 between the layers 32 and 34 of the adhesive base 110, as described above and shown in the figures, the housing 130 is securely coupled to and within the adhesive base 110. Also, the bottom layer 34 and the adhesive layer 36 are each structured to extend to at least beyond the inner edge of the inner lip 74 of the housing 130. In certain embodiments, the bottom layer 34 and the adhesive layer 36 are each structured to extend to at least the inner edge of the lip 44 of the port guard 120. Such a structure prevents the housing 130 and the port guard 120 from gouging or otherwise contacting the user's skin.

As shown in FIG. 14, the port guard 120 can then be placed within the housing 130. The lip 44 of the port guard 120 may contact the inner lip 74 of the housing 130 and rest thereon. Alternatively, a cushion layer 64 may be coupled to the underside of the lip 44 and the cushion layer 64 may contact the inner lip 74 of the housing 130 and rest thereon, as shown in FIG. 14. The lip 44 also rests under the protrusions 76, described above and shown in FIG. 9, the protrusions 76 keeping the lip 44 in place on the inner lip 74 to prevent the port guard 120 from moving in the vertical direction.

The components defining any of the adhesive base 110, the guard 100, the port guard 120, and the housing 130 may be formed of any of many different types of materials or combinations thereof that can readily be formed into shaped objects provided that the components selected are consistent with the intended operation of a guard and adhesive base for protecting an implantable port. For example, all of the components should be formed of latex-free materials so as to not irritate the user's skin. Moreover, the adhesive base can be formed of flexible materials, such as woven material with fibers embedded therein. Also, the guard 100, the port guard 120, and the housing 130 can be formed of rigid, durable materials, such as plastic, that are able to withstand repetitive abrading and rubbing, as well as frequent impacts received from mild contact sports. However, the various components may also be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

One of the methods of using the guard 100 or 120 to protect the implantable port can include placing the guard 100 or 120 over the implantable port and securing the guard 100 or 120 to the user's skin over the implantable port by adhesion such that the guard 100 or 120 does not contact the user's skin above or immediately around the implantable port.

Another method of using the guard 100 or 120 to protect the implantable port can include coupling the adhesive base 110 to the guard 100 or 120 prior to adhering the adhesive base 110 to the user's skin, such that the guard 100 or 120 is held in place over the implantable port by the adhesive base 110, but the guard 100 or 120 does not contact the user's skin above or immediately around the implantable port.

Another method of using the guard 120 to protect the implantable port can include coupling the housing 130 to the user's skin by adhesion. Thereafter, the user can couple the port guard 120 to the housing 130 by placing his/her fingers of one hand on the tabs 86 to secure the housing 130 in place. The user can then use the fingers of his/her other hand to slide the w-shaped nose portion 48 of the port guard 120 into the corresponding w-shaped receiving portion 78 on the housing 130 such that the lip 44 on the front end 50 of the port guard 120 contacts the elastic member 90 in the groove 75 of the housing 130. Once the w-shaped nose portion 48 is docked in the w-shaped receiving portion 78, the port guard 120 should not shift side to side. Moreover, the lip 44 of the port guard 120 is secured in the vertical direction between the inner lip 74 and the protrusions 76. In certain embodiments, where the port guard 120 is made of semi-rigid, but flexible material, the user can place his/her fingers of the other hand on the holes 56 of the port guard 120 and squeeze his/her fingers together to deform the port guard 120. By deforming the port guard 120, the user may compress the port guard 120 to the point that the docking tabs 54 of the port guard 120 are able to fit into the notches 77 of the housing 130 and contact the elastic members 90 in the back end 84 of the housing 130.

Thereafter, upon release of the holes 56, the flexible port guard 120 will return to its normal shape and, consequently, the lip 44 on the front end 50 of the flexible port guard 120 will apply pressure to and deform the elastic member 90 in the front end 82 of the housing 130 and the docking tabs 54 will apply pressure to and deform the elastic members 90 that are in the back end 84 of the housing 130, such that the pressure from the elastic members 90 in the housing 130 will secure the port guard 120 in position. Moreover, as the docking tabs 54 compress the elastic members 90, the lip 44 on the back end 52 of the port guard 120 slips between the inner lip 74 and the protrusions 76 on the back end 84 of the housing 130 and further secures the port guard 120 to the housing 130.

In certain other embodiments, where the port guard 120 is made of rigid, nonflexible material, the user can couple the port guard 120 made of rigid material to the housing 130 by placing, similarly to the port guard 120 made of flexible material, his/her fingers of one hand on the tabs 86 to secure the housing 130 in place and use the fingers of his/her other hand to slide the w-shaped nose portion 48 of the non-flexible port guard 120 into the corresponding w-shaped receiving portion 78 on the housing 130 such that the lip 44 of the port guard 120 contacts the elastic member 90 in the groove 75 of the housing 130. However, unlike the port guard 120 made of flexible material, the user places his/her fingers of the other hand on the holes 56 of the port guard 120 to grip the port guard 120 firmly enough to push the port guard 120 into the elastic member 90 in the groove 75 of the housing 130 to deform the elastic member 90. By deforming the elastic member 90, the user can shift the rigid port guard 120 upward toward the front end 82 of the housing 130 enough to place the docking tabs 54 of the rigid port guard 120 into the notches 77 of the housing 130 such that the docking tabs 54 contact the elastic members 90 in the back end 84 of the housing 130.

Thereafter, upon release of the holes 56, the elastic member 90 in the front end 82 of the housing 130 will push back against the lip 44 in the front end 50 of the rigid port guard 120 to apply force to the rigid port guard 120 to make the docking tabs 54 in the back end 52 of the rigid port guard 120 apply pressure to and deform the elastic members 90 that are in the back end 84 of the housing 130, such that the pressure from the elastic members 90 in the front end 82 and back end 84 of the housing 130 will secure the port guard 120 in position. Moreover, when the docking tabs 54 compress the elastic members 90 in the back end 84 of the housing 130 due to the elastic member 90 in the front end 82 pushing against the rigid port guard 120, the lip 44 on the back end 52 of the port guard 120 slips between the inner lip 74 and the protrusions 76 on the back end 84 of the housing 130 and the port guard 120 is thus further secured to the housing 130.

When properly coupled to the housing 130, the port guard 120 (made of either flexible material or rigid material) does not contact the user's skin, nor does the port guard 120 contact the implantable port embedded under the user's skin. Moreover, when properly coupled to the housing 130, the port guard 120 can help prevent any object from contacting the implantable port or the skin of the user above or immediately around the implantable port.

To remove the port guard 120 from the housing 130, the above-described steps are simply reversed. The user grips the holes 56 in the port guard 120 and either deforms the port guard 120 or the elastic member 90 in the front end 82 of the housing 130 to release the docking tabs 54 from the elastic members 90 embedded in the frame 70 on the back end 84 of the housing 130. After the docking tabs 54 are released, the user may remove the w-shaped nose portion 48 of the port guard 120 from the corresponding w-shaped receiving portion 78 of the housing 130 and remove the port guard 120 from the housing 130.

Another method of using the guard to protect the implantable port can include coupling the adhesive base 110 to the housing 130 prior to adhering the adhesive base 110 to the user's skin with the housing 130 attached thereto. In such a configuration, the housing 130 is held in place over the implantable port by the adhesive base 110 and the housing 130 does not contact the skin above or on the side of the implantable port. Thereafter, the port guard 120 can be releasably coupled to the housing 130 as described above.

Prior to attaching either the guard 100, the adhesive base 110, or the housing 130 to the user's skin, the user may clean the area around the implantable port to ensure better adhesion between the adhesive layer 36 and the skin. The adhesive layer 36 has been described as being attached to the underside of the bottom layer 34 of the adhesive base 110 to couple the adhesive base 110 to the user. Alternatively, the adhesive layer 36 may be used in conjunction with the guard 100 and the housing 130 to attach each to the user's skin, as desired. Indeed, any of the guard 100 and the housing 130 may have the adhesive layer 36 attached directly to the underside thereof to allow each of the guard 100, and the housing 130 to individually attach to the user's skin. After cleaning the skin around the implantable port, the user may attach the adhesive base 110, the guard 100, or the housing 130 to the skin.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A guard for an implantable port, comprising:
a cover,
wherein the cover is structured to be placed over the implantable port to substantially cover the implantable port.

2. The guard of claim 1, the guard further comprising:
an adhesive base having a hollow center portion therein,
wherein the cover is releasably coupled to the adhesive base, and the adhesive base is structured to releasably adhere to the user around the implantable port, allowing the cover to substantially cover the implantable port without the cover contacting the user's skin above the implantable port.

3. The guard of claim 1, the guard further comprising:
a peripheral edge defining the outer circumference of the rounded cover;
an opening in the peripheral edge; and
a lip extending at an angle from the peripheral edge,
wherein the cover has a rounded shape, and
wherein the lip releasably couples to the user around the implantable port, allowing the cover to substantially cover the implantable port without the cover coming into contact with the user's skin above the implantable port.

4. The guard of claim 3, the guard further comprising:
a top surface in the rounded cover; and
a hole in the rounded cover,
wherein the top surface is substantially flat.

5. The guard of claim 2, the guard further comprising:
the cover having a rounded triangular shape;
a peripheral edge defining the outer circumference of the rounded cover;
an opening in the peripheral edge; and
a lip extending at an angle from the peripheral edge,
wherein the lip releasably couples to the adhesive base.

6. The guard of claim 5, the guard further comprising:
underside and topside layers of the adhesive base; and
an adhesive layer on the bottom of the underside layer,
wherein a portion of the topside layer is fixedly coupled to a portion of the underside layer,
wherein a remaining portion of the topside layer is structured to be placed on and couple to an upper side of the lip and a remaining portion of the underside layer is structured to be placed under and couple to a bottom side of the lip to releasably couple the lip of the cover to the adhesive base, and
wherein the adhesive layer releasably adheres the adhesive base to the user.

7. The guard of claim 3, the guard further comprising:
a top surface of the rounded cover; and
a hole in the top surface,
wherein the peripheral edge bows outward from the center of the triangular shape and the lip runs along the entire peripheral edge, except for the opening, the lip extending from the peripheral edge at an angle that is parallel with the top surface.

8. The guard of claim 6, the guard further comprising:
a cushioning layer fixedly coupled to the underside of the lip, wherein the cushioning layer is positioned between the lip and the remaining portion of the underside layer under the condition that the cover is coupled to the adhesive base.

9. A guard for an implantable port, comprising:
a triangular, dome-shaped cover;
a peripheral edge defining the outer circumference of the triangular cover;
an opening in the peripheral edge; and
a lip extending at an angle from the peripheral edge.

10. The guard of claim 9, the guard further comprising:
a top portion in the cover that is substantially flat;
front and back ends of the cover; and
a plurality of holes in the cover,
wherein the peripheral edge bows outward from the center of the cover,
wherein the opening is arc-shaped,
wherein the lip extends from the peripheral edge parallel to the top portion, and
wherein one of the plurality of holes is positioned in the front end of the cover above the peripheral edge and below the top portion and another of the plurality of holes is positioned in the back end of the cover above the peripheral edge and below the top portion.

11. The guard of claim 9, further comprising:
a housing having a hollow center portion therein, the housing further comprising:
a triangular-shaped frame defining the hollow center portion;
an outer flange extending outwardly from the frame;
an inner flange extending inwardly from the frame;
a gap in the frame; and
a plurality of projections extending inwardly from the frame and above the inner flange,
wherein the housing is placed around the implantable port and the cover is releasably coupled to the housing.

12. The guard of claim 11, wherein the cover is releasably coupled to the housing by placing the lip of the cover between the projections and the inner flange to secure the lip between the projections and the inner flange.

13. The guard of claim 12, further comprising:
a w-shaped nose portion in a front end of the cover where the peripheral edge contacts the lip; and
a w-shaped receiving portion in the housing for receiving the w-shaped nose portion of the cover;
a docking tab extending from the lip at a back end of the cover; and
elastic members coupled to the frame between the projections and the inner flange, wherein one elastic member is positioned on a front end of the frame under the w-shaped receiving portion and deforms under the condition that the lip on the front end of the cover is pressed into the one elastic member, and wherein another elastic member is positioned in the frame on a back end of the frame and deforms under the condition that the docking tab on the back end of the cover is pressed into the another elastic member.

14. The guard of claim 11, further comprising:
an adhesive base having a hollow center portion therein, wherein the adhesive base is releasably coupled to the housing such that the hollow center portion of the housing overlaps the hollow center portion of the adhesive base, the adhesive base being structured to releasably adhere to the user around the implantable port such that implantable port is positioned within the frame of the housing, the housing allowing the cover to substantially cover the implantable port without the cover contacting the user's skin above the implantable port under the condition that the cover is releasably coupled to the housing.

15. The guard of claim 14, further comprising:
underside and topside layers of the adhesive base; and
an adhesive layer on the bottom of the underside layer,
wherein a portion of the topside layer is fixedly coupled to a portion of the underside layer,
wherein a remaining portion of the topside layer is structured to be placed on and couple to the outer flange of the housing and a remaining portion of the underside layer is structured to be placed under and couple to the housing to releasably couple the housing to the adhesive base, and
wherein the adhesive layer releasably adheres the adhesive base to the user.

16. The guard of claim 15, further comprising:
a cushioning layer fixedly coupled to the underside of the housing, wherein the cushioning layer is positioned between the housing and the remaining portion of the underside layer under the condition that the housing is coupled to the adhesive base.

17. The guard of claim 15, wherein the cover is releasably coupled to the housing by positioning the lip of the cover between the projections and the inner flange to secure the lip between the projections and the inner flange.

18. A method of protecting an implantable port comprising:
placing a cover over the implantable port to protect the implantable port; and
securing the cover over the implantable port.

19. The method of claim 17, wherein the placing a cover over the implantable port further comprises:
placing a detachable housing around the implantable port; and
coupling the cover to the detachable housing, and
wherein the securing the cover over the implantable port further comprises:
adhering the detachable housing to the user's skin around the implantable port such that neither the detachable housing nor the cover contacts the user's skin above the implantable port.

20. The method of claim 17, wherein the placing a cover over the implantable port further comprises:
coupling an adhesive base to a detachable housing;
orienting the adhesive base and the detachable housing coupled thereto around the implantable port so that the adhesive base and the detachable housing do not contact the user's skin above the implantable port; and
coupling the cover to the detachable housing, and
wherein the securing the cover over the implantable port further comprises:
adhering the adhesive base to the user's skin around the implantable port such that none of the adhesive base, the detachable housing, or the cover contacts the user's skin above the implantable port.
